(19) United States
(12) Patent Application Publication
(10) Pub. No.: US 2024/0374212 A1
(43) Pub. Date: Nov. 14, 2024

(54) SYSTEM FOR MONITORING A VITAL SIGN

(71) Applicant: SMARTCARDIA SA, Lausanne (CH)

(72) Inventors: Rubén BRAOJOS LÓPEZ, Chavannes-près-Renens (CH); Srinivasan MURALI, Lausanne (CH); Francisco Javier RINCON VALLEJOS, Renens (CH)

(21) Appl. No.: 18/285,962

(22) PCT Filed: Apr. 7, 2021
(86) PCT No.: PCT/IB2021/052902

§ 371 Date: Oct. 6, 2023

(51) Int. Cl. A61B 5/00; A61B 5/257; A61B 5/282; A61B 5/346

(52) U.S. Cl. CPC A61B 5/684; A61B 5/257; A61B 5/282; A61B 5/346; A61B 5/6823; A61B 5/6833; A61B 5/7246; A61B 2560/0214; A61B 2560/0223; A61B 2560/0443; A61B 2560/0468; A61B 2562/0219; A61B 2562/22; A61B 2562/227

(57) ABSTRACT

A system for monitoring a vital sign of a living organism, wherein the system comprises a patch device configured to be placed on the living organism, wherein the patch device comprises a sensor means configured to provide sensor data, is configured to determine a spatial orientation of the patch device by means of the sensor data when the patch device is placed on the living organism.

### TECHNICAL FIELD

The disclosure relates to a system for monitoring a vital sign of a living organism. Furthermore, the disclosure relates to a system, two methods, a computer program and a computer-readable medium and a method for using a system.

### BACKGROUND

In modern medicine, monitoring body functions, such as the activity of the heart or the oxygen saturation of the blood, are very important and powerful possibilities for gaining insight into the proper functioning of living organisms, for example humans or animals and for detecting possible health problems.

Traditionally, electrocardiograms (abbreviated ECG) have long been recorded by attaching electrodes to the body of a patient and by linking these electrodes to a computer configured to record the ECG by means of a multitude of cables. Likewise, oxygen saturation measurements have for a long time traditionally been carried out by finger clips or the like which were connected to a measuring device or computer by means of a cable.

In recent years, wearable devices for monitoring body functions, for example wearable devices for monitoring the functioning of the heart and also for monitoring oxygen saturation in the blood of living organisms, have become available. In particular, such wearable devices have become available in the form of patches which comprise electronic measuring circuits and which can be attached to the chests of patients for example. Typically, such patches do not have to be connected to a recording infrastructure via cables but are rather able to send data to a recording infrastructure or an analysis infrastructure wirelessly.

One problem with such wearable patches, in particular patches for ECG measurements, is that the preciseness of the data acquired by such patches depends on the accuracy of the placement of the patches on the bodies of the living organisms which are to be monitored. In other words: Such patches can only supply sufficiently precise results if they are correctly placed on the body of a patient.

Furthermore, most patches are not very flexible because they have predefined and standard dimensions. This is problematic because there is a large variety of body sizes in patients. For example, a patch which is well adapted to the body dimensions of a normally weighted adult will not necessarily also function well when placed on the body of a child. The reason for this is that, especially when ECG measurements are to be carried out, the electrode surface areas of the different leads of the ECG should be located at precise body locations in order to yield good measurement results. If a patch with a certain dimension adapted to an adult is put on a child, the electrode surface areas will not be at the right locations and the measurement result will be inaccurate, at least to a certain extent.

Furthermore, the medical monitoring patches available up to now often have the problem that they are not very flexible. For example, a patch is typically configured to record an ECG with a predefined number of leads but it is not necessarily possible to record ECGs with different numbers of leads with one and the same patch.

### SUMMARY

It is an object of the disclosure, per an embodiment, to solve or to at least diminish the above-mentioned disadvantages.

This problem is solved by a system, per an embodiment, for monitoring a vital sign of a living organism, wherein the system comprises a patch device configured to be placed on the living organism, wherein the patch device comprises a sensor means configured to provide sensor data, wherein the system is configured to determine a spatial orientation of the patch device by means of the sensor data when the patch device is placed on the living organism. “Monitoring a vital sign” can for example correspond to the monitoring of the functioning of a heart, in particular the recording of an electrocardiogram (ECG) or of an electromyogram or of another body parameter such as, for example, a blood oxygen saturation. A “living organism” can be a human being or yet an animal such as a dog, a horse or another animal. In typical embodiments, the “sensor means” comprises at least one sensor, for example an accelerometer and/or a gyroscope. The expression “placed on the living organism” refers to attaching the patch device on the body of the living organism, for example by means of an adhesive surface. In embodiments, the “spatial orientation” is typically a rotation angle, preferably a rotation angle around an axis that points out from a chest of the living organism, preferably perpendicularly. This embodiment of the system solves the above-mentioned problem because it makes it possible to gain an insight in how precisely the patch device has been placed on the body of the living organism and to draw conclusions from this information, for example by taking into account the precise spatial orientation when analysing electric potentials used for ECG recordings.

The problem is furthermore solved by a system, per an embodiment, for monitoring a vital sign of a living organism, wherein the system comprises a patch device configured to be placed on the living organism, wherein the patch device is configured to be adapted to multiple use scenarios. In this regard, the expression “use scenario” is to be understood such that it refers to for example to use on human bodies of different morphology (e.g. skinny bodies, normal-weight bodies, obese bodies, tall bodies, small bodies and so on) and/or to use with a variable number of active electrodes and/or to use with a variable number of active ECG leads. This embodiment of the system solves the above-mentioned problem because the possible adaptation to multiple use scenarios makes the system and in particular the patch device more flexible.

The following additional explanations concerning embodiments in principle apply to both systems, namely the system in which the patch device comprises the sensor means and the system in which the patch device is configured to be adapted to multiple use scenarios. In particular, the following explanations concerning a patch device relate to the patch devices in both these systems. In particular, the patch devices of both these systems can each comprise one or more of the features outlined hereafter.

In embodiments, the patch device comprises a disposable patch component and an electronic component, wherein the electronic component comprises the sensor means and/or wherein the electronic component is configured to determine said spatial orientation of the patch device. Having a patch with a disposable patch component on one hand and an electronic component on the other hand has the advantage that after use of a patch only a part of the patch has to be thrown away and the electronic component can be reused. The fact of integrating the sensor means in the electronic component is advantageous because like this, the sensor means can be reused. It is, however, also possible to use a patch device with an integrated form (i.e. a patch device having electronics integrated in it) that does not make a clear difference between a disposable part and a reusable part.

In embodiments, the patch device, preferably the disposable patch component, comprises a main body and an arm portion, wherein the main body comprises a housing for the electronic component, and/or wherein the arm portion has an at least partially rounded form, and/or wherein the main body comprises at least one, typically at least two electrode contact areas, and/or wherein the arm portion comprises at least one, typically at least two electrode contact area(s), and/or wherein the patch device is configured in such a way that the system can acquire an electrocardiogram of a living organism by means of the electrode area(s). The main body typically has essentially the form of a plaster, typically with rounded edges. The arm portion can correspond to a section of a circle, for example approximately a quarter of a circle. The arm portion is typically attached to one side of the main body. In embodiments, the arm portion is rounded in a direction that corresponds to the direction of an imaginary line drawn from the side of the main body to which the arm portion is attached to the other side of the main body. In embodiments, when the patch device is placed such that the length of the main body is aligned with the horizontal direction, the arm portion extends below the main body, at least partially. The arm portion is typically rounded such that a length of the main body corresponds approximately to the radius of a circle that one would obtain if one would transform the approximate quarter of a circle that the arm portion builds into a full circle. Such a configuration with main body and arm portion has the advantage of making it possible to provide a patch for ECG measurements which can be adapted to various body sizes, for example by means of a rotation, depending on the size of the body to which the patch device is to be attached. Using two electrode contact areas on the main body and two electrode contact areas on the arm portion makes it possible to adequately distribute electrode contact areas, for example for recording a 7-lead ECG. The expression “electrode contact area” is to be understood in the sense of an electrode pad, which is configured to be in contact with a skin of the living organisms in order to sense electrical activities emitted by the heart of the living organism. These electrode contact areas can also be referred to simply as ECG electrodes.

In embodiments, the arm portion is configured to be detached from the main body, wherein the patch device comprises a perforation arranged between the arm portion and the main body for detaching the arm portion from the main body. Such a possibility for detaching the arm portion from the main body makes the patch device particularly flexible, because in cases where the arm portion is not needed, for example if no 7-lead ECG has to be recorded, the arm portion can simply be detached and the patch can therefore be used in a much more compact and comfortable to wear configuration. In other words, the possibility to detach the arm portion from the main body transforms the patch device into a more flexible patch device configured to be used in different use scenarios. The inventors have found that in particular a perforation at the location where the arm portion is attached to the main body of the patch device is a convenient way to enable a detachment of the arm portion from the main body because it makes it possible to carry out the detaching simply by means of pulling on the two components. Such a perforation is therefore a simple and yet effective and convenient way to enhance the flexibility of the patch device.

In embodiments, the patch device, preferably the main body, comprises at least two batteries, wherein the batteries are typically arranged symmetrically around a centre of the patch device, preferably around a centre of the main body, and/or wherein the batteries are typically located at two opposite ends of the patch device, preferably at two opposite ends of the main body, and/or wherein the batteries are connected in parallel, and/or wherein the patch device, preferably the main body, comprises a positive voltage pin and a negative voltage pin, wherein the voltage pins are configured to connect to the electronic component, and/or wherein at least two batteries, preferably all batteries, are connected to the positive voltage pin and/or wherein at least two batteries, preferably all batteries, are connected to the negative voltage pin. The fact of arranging the batteries in parallel and furthermore symmetrically on the patch is advantageous because it can lead to a longer lifetime of the batteries. Letting at least two batteries or even all batteries use the positive and negative pins has the advantage, per embodiments, that it makes it possible to minimise the size of the electronic component and of the patch device because less pins are needed than in a case where every battery would have its own positive and negative pins. However, other configurations for the batteries are of course also possible.

In embodiments, the patch device, preferably the disposable patch component, comprises a network of electrode connection lines for connecting the electrode contact areas to the electronic component, and/or wherein the electrode connection lines are arranged in a snake coil form on the arm portion. In embodiments, the electrode connection lines feed into electrode connection pins integrated in the disposable patch component so that the electronic component can be connected to these electrode connection pins. Arranging the electrode connection lines in a snake coil form on the arm portion has the advantage, per embodiments, that the arm portion can in principle be stretched, at least to a certain extent. Furthermore, the inventors have found that the snake coil form helps to avoid problems of electromagnetic compatibility. However, the electrode connection lines can also simply be essentially straight.

In embodiments, the electrode connection lines comprise a mixture of silver and silver chloride, wherein the mixture preferably comprises approximately 50% of silver and approximately 50% of silver chloride, more preferably approximately 80% of silver and approximately 20% of silver chloride, yet more preferably approximately 95% of silver and approximately 5% of silver chloride. Throughout this specification, the expression “approximately” is to be understood as referring to an allowable range around a certain value of for example ±20%, preferably ±10%, more preferably ±5%. Using a mixture of silver and silver chloride for the electrode connection lines has the advantage of making it possible to reduce the cost of the electrode connection lines because silver chloride is less expensive than silver. The inventors have found that use of silver chloride instead of silver is acceptable for the purposes of the transmission of ECG signals and the values mentioned above do not have a significant negative influence on the quality of the ECG recordings.

In embodiments, the patch device, preferably the disposable patch component, comprises a network of battery connection lines for connecting the batteries to the positive voltage pin and the negative voltage pin, wherein the battery connection lines preferably comprise at least approximately 100% silver. The use of at least almost pure silver for the battery connection lines has the advantage of minimising resistive losses in the battery connection lines, thereby improving the lifetime of the batteries.

In embodiments, the battery connection lines have a larger cross section surface than the electrode connection lines. The expression “larger cross section surface” means that the battery connection lines are larger and/or thicker and/or wider and/or higher than the electrode connection lines. The inventors have found that a certain size of a cross section surface of the battery connection lines offers a good trade-off between cost on one hand and lifetime of the batteries on the other hand but that this particular size of cross section surface is not necessarily needed for the electrode connection lines. In other words, the electrode connection lines can have smaller cross section surfaces than the battery connection lines without having a negative influence on the quality of the ECG recordings. By choosing such dimensions, the cost of the patch device can be reduced.

In embodiments, the patch device, typically the main body and/or the arm portion, comprise(s) a multi-piece protective cover on an adhesive surface on its backside(s). The “backside” of the patch device is the side of the patch device which faces the living organism. In other words: The side of the patch device which touches the skin of the living organism when the patch is attached to the living organism is the backside of the patch device. The adhesive surface on the backside is typically formed by a glue or other adhesive which is configured to stick to skin. The use of a multi-piece protective cover has the advantage that it is possible to only let certain parts of the patch device be in contact with the skin of the living organism. Like this, the flexibility of the system is increased. In embodiments, the multi-piece protective cover comprises a separate protective cover piece for at least one of the electrode contact areas, preferably one single protective cover piece for each electrode contact area. In other words, typically every electrode contact area has its own protective cover piece. This makes it possible to precisely activate only those electrodes which are desired in a particular use scenario. Such a multi-piece protective cover therefore strongly improves the flexibility of the system.

In embodiments, the system comprises a remote device, wherein the remote device is configured to participate in the determination of said spatial orientation of the patch device, wherein the remote device is preferably configured to determine said spatial orientation of the patch device solely based on the sensor data. In embodiments, the remote device is configured to determine said spatial orientation of the patch device without additional help from the electronic component of the patch device. In other embodiments, however, it is also possible that no remote device is being used and that the electronic component does the determination of the spatial orientation of the patch device all by itself.

The problem is furthermore solved by embodiments of a method for determining a spatial orientation of a patch device for monitoring a vital sign of a living organism during a placing of the patch device on the living organism, wherein the method comprises a sensor data analysis step during which sensor data supplied by a sensor means of the patch device is analysed in order to determine the spatial orientation. In embodiments, the patch device used in the method is a patch device as previously described. In this context, it is important to understand that the above-mentioned system can actually consist of a patch device as explained. In this case, the patch device would therefore be the system. However, in situations where also a remote device is being used, the system would comprise a patch device and the remote device and possibly other components. The method is typically computer-implemented.

In embodiments, the method comprises a baseline calibration step, during which the patch device is provisionally positioned on the living organism in a predefined baseline position and baseline sensor data is recorded, and the method comprises a rotating step during which the patch device is rotated around an imaginary rotation axis sticking out of the living organism, preferably at least approximately perpendicularly, until a desired orientation of the patch device is reached, wherein the rotating step is followed by the sensor data analysis step, wherein during the sensor data analysis step, sensor data recorded at the desired orientation is compared with the baseline sensor data in order to determine the spatial orientation. The combination of such steps may be advantageous, per embodiments, because it leads to a very good precision of the determination of the spatial orientation of the patch device. A “desired orientation of the patch device” is typically an orientation which corresponds to an optimal placing of the patch device on the particular body of the living organism, for example a particular positioning of the patch device on a child's body.

The problem is furthermore solved by embodiments of a method for recording an ECG of a living organism, wherein the method comprises a method for determining a spatial orientation of a patch device according to any of the embodiments presented above, wherein the method for recording comprises a matching process during which the spatial orientation determined during the sensor data analysis step is used for matching obtained electrocardiogram signals with pre-recorded electrocardiogram data, and/or wherein the method for recording comprises an angle adaptation step during which at least one ECG angle, preferably all ECG angles, is/are determined based on the spatial orientation determined during the sensor data analysis step, and/or wherein the method for recording comprises a lead renaming step during which at least one ECG lead, preferably all ECG leads, is/are renamed based on the spatial orientation determined during the sensor data analysis step, and/or wherein the method for recording comprises a chest-arm-detection step during which, based on the spatial orientation determined during the sensor data analysis step, it is automatically detected whether the patch device, preferably the main body, is placed on a chest or on an arm of a human being. It is self-evident that the method for recording typically also includes a step of placing and/or attaching the patch device on a body of a human being and/or starting a recording of the ECG ones all desired preliminary steps have been carried out.

A computer program comprises, in an embodiment of the disclosure, instructions which, when the program is executed by a computer, cause the computer to carry out a method according to any of the above-mentioned embodiments. The expression “computer” is to be understood as referring to any device or structure that is able to execute the instructions. The computer program can also be referred to as computer program product.

A computer-readable medium comprises, in an embodiment of the disclosure, computer program code for carrying out a method according to any of the above-mentioned embodiments and/or comprises a computer program according to the above-mentioned embodiment. The expression “computer-readable medium” can be understood as referring in particular but not exclusively to hard disks and/or servers and/or memory sticks and/or flash drives and/or DVDs and/or Blu-ray disks and/or CDs. Furthermore, the expression “computer-readable medium” can also refer to a data stream which is for example established when a computer program and/or a computer program product is downloaded from the internet.

A method for using a system according to any of the above-mentioned embodiments comprises attaching the main body of the patch device to an upper arm of a patient and attaching the arm portion of the patch device to a chest of the patient, wherein the main body comprises the electronic component. In such a method, the patch device and/or the main body is preferably attached to the upper arm of the patent by means of an adhesive and/or by means of a band, wherein the band is typically a textile band and/or an elastic band and/or a plastic band. In embodiments, the method for using the system comprises-in particular during a positioning of the patch device on the arm-determining the spatial orientation, preferably the spatial rotation, of the patch device and automatically deducting from the spatial orientation and/or rotation whether the patch device is placed on the chest or the arm of the patient.

### BRIEF DESCRIPTION OF THE FIGURES

In the following, the disclosure is described in detail by means of a drawing, wherein shows:

FIG. 1: A schematic view of a patch device according to an embodiment,

FIG. 2: another schematic view of the patch device already shown in FIG. 1 with horizontal distances between the different electrode contact areas indicated,

FIG. 3: a zoom-in on the patch device already shown in FIGS. 1 and 2,

FIG. 4: another zoom-in on a detail of the patch device shown in FIGS. 1 and 2,

FIG. 5: a patch device according to one embodiment placed on a chest of a human being, and

FIG. 6: a block diagram visualising a method according to one embodiment.

### DETAILED DESCRIPTION

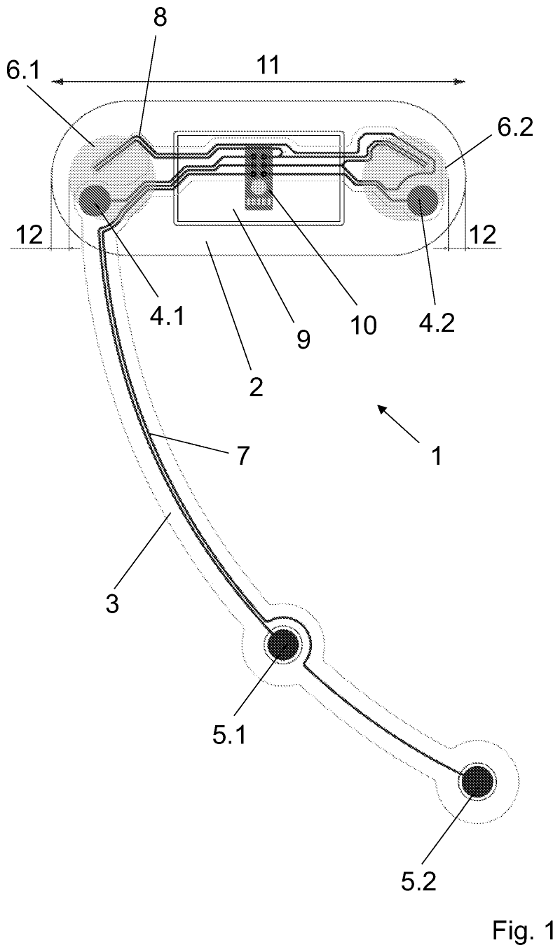

FIG. 1 shows a schematic view of a patch device according to an embodiment. In particular, FIG. 1 shows a patch device 1 of a system for monitoring a vital sign of a living organism. The patch device 1 is shown in a transparent representation, or in other words: x-ray-like. The patch device 1 comprises a main body 2 and an arm portion 3. The arm portion 3 is attached to one side of the main body 2, has an at least partially rounded form and follows a path below the main body 2. The patch device 1 furthermore comprises two electrode contact areas 4.1, 4.2 in its main body 2 and two electrode contact areas 5.1, 5.2 in its arm portion 3. The electrode contact areas 4.1, 4.2, 5.1, 5.2 are configured to be in touch with a living organism's skin when the patch device 1 is placed on the living organism. When the patch device 1 is used for ECG measurements, the electrode contact areas 4.1, 4.2, 5.1, 5.2 can also be referred to as ECG electrodes. The patch device 1 furthermore comprises two batteries 6.1, 6.2, which are arranged on opposite sides of the main body 2 of the patch device 1. The patch device 1 furthermore comprises a network of electrode connection lines 7. The electrode connection lines connect the electrode contact areas 4.1, 4.2, 5.1, 5.2 to corresponding pins on a contact board 10 arranged inside a housing 9. The housing 9 is configured to house an electronic component of the patch device 1, which is, however, not explicitly shown in FIG. 1. One can therefore say that the patch device 1 shown in FIG. 1 is actually the disposable patch component because the electronic component is not shown in FIG. 1. The contact board 10 is configured to be in contact with the electronic component when the latter is being placed in the housing 9. The patch device 1 furthermore comprises a network of battery connection lines 8. The network of battery connection lines 8 connects the two batteries 6.1, 6.2 to corresponding voltage pins on the contact board 10. The batteries 6.1, 6.2 are connected in parallel. Both batteries 6.1, 6.2 share a common negative voltage pin and a common positive voltage pin on the contact board 10 (for reasons of better conciseness, the pins on the contact board 10 are not equipped with reference signs). The main body 2 of the patch device 1 has a main body length 11. In typical embodiments, the main body length 11 equals approximately 120 mm. A distance between the batteries 6.1, 6.2 from an edge of the main body 2 of the patch device 1 is referred to as main body edge width 12. In typical embodiments, the main body length 11 equals approximately 5 mm.

In embodiments, the housing 9 houses an electronic component, wherein the electronic component comprises a sensor means configured to provide sensor data, wherein the sensor means for example comprises an accelerometer and/or a gyroscope. Based on the sensor data, it is preferably possible to determine a spatial orientation of the patch device 1 when the patch device 1 is placed on the living organism.

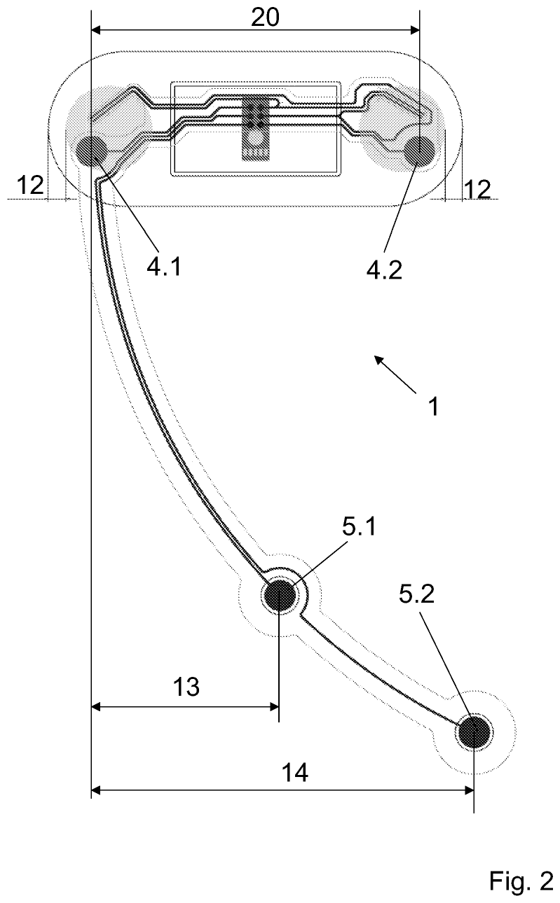

FIG. 2 shows another schematic view of the patch device already shown in FIG. 1 with horizontal distances between the different electrode contact areas indicated. In particular, FIG. 2 shows the same patch device 1 as already shown in FIG. 1 but there are different reference signs shown in FIG. 2. In particular, some reference signs which are not relevant to the following explanations have been omitted and additional reference signs, which are of relevance for the following explanations, have been added. In particular, a main body electrode contact area distance 20 is indicated in FIG. 2. The main body electrode contact area distance 20 measures the distance between the centres of the two electrode contact areas 4.1, 4.2 of the main body. Also, a first horizontal offset 13 which measures the distance in the direction of the length of the main body between the electrode contact area 4.1 (this electrode contact area is also referred to as first electrode contact area of the main body) and the electrode contact area 5.1 of the arm portion (this particular electrode contact area is also referred to as first electrode contact area of the arm portion). In FIG. 2 is furthermore indicated a second horizontal offset 14 which measures the distance in the direction of the length of the main body between the first electrode contact area 4.1 of the main body and a second electrode contact area 5.2 of the arm portion. The second electrode contact area 5.2 of the arm portion is located at an end of the arm portion. The first electrode contact area 5.1 of the arm portion is located/arranged between the first electrode contact are 4.1 of the main body and the second electrode contact area 5.2 of the arm portion. The first electrode contact area 5.1 of the arm portion is located underneath the main body in the case of FIG. 2 where the main body is oriented horizontally. The first horizontal offset 13 equals approximately 40-60%, preferably approximately 50% of the second horizontal offset 14. The second horizontal offset 14 is approximately 16% longer than the main body electrode contact area distance 20.

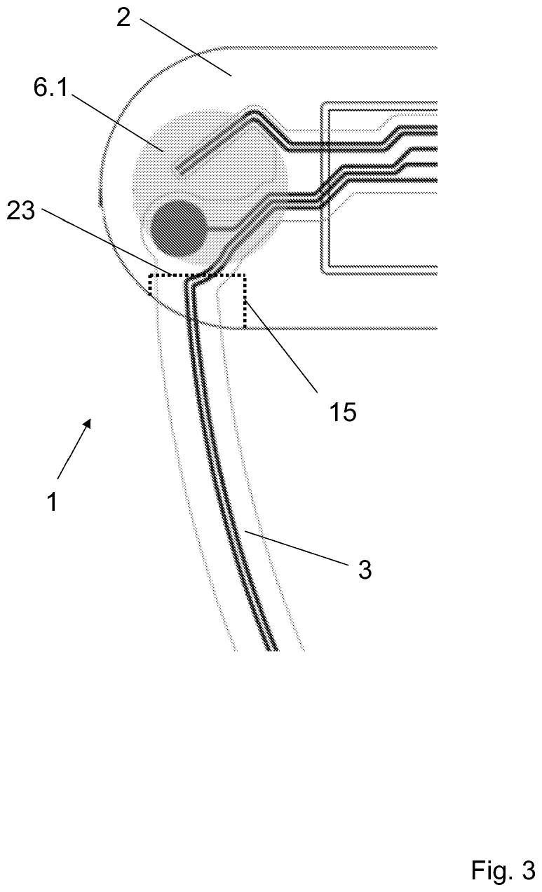

FIG. 3 shows a zoom-in on the patch device 1 already shown in FIGS. 1 and 2. In particular, the zoom-in displayed in FIG. 3 shows a detail that was not yet shown in FIGS. 1 and 2, namely a perforation 15. The perforation 15 makes it possible to detach the arm portion 3 of the patch device 1 from the main body 2 of the patch device 1 in a very convenient manner. In particular, the perforation 15 makes it possible to detach the arm portion 3 from the main body 2 by simply holding the main body 2 and pulling firmly on the arm portion 3. The perforation 15 comprises a perforation top edge 23. A distance between the first battery 6.1 and the perforation top edge 23 equals between 5 and 1 mm, preferably between 4 and 2 mm. The inventors have found that such a distance between the first battery 6.1 and the perforation top edge 23 is advantageous, per embodiments, because on one hand it keeps a sufficient distance from the battery 6.1 and on the other hand it minimises an open loop of the electrode connection lines, thereby minimising negative effects of electromagnetic compatibility when the arm portion 3 is detached from the main body 2.

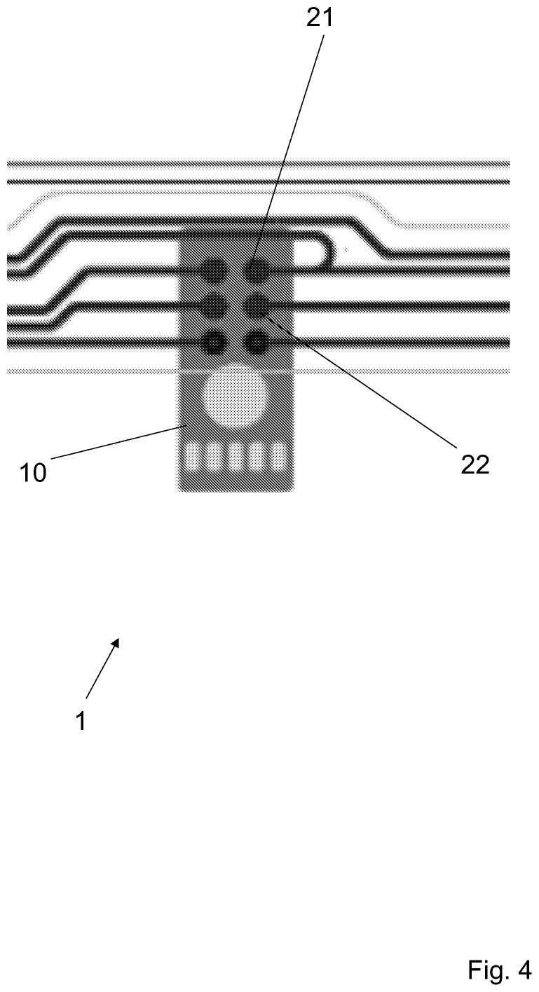

FIG. 4 shows another zoom-in on a detail of the patch device 1 shown in FIGS. 1 and 2. In particular, the zoom-in in FIG. 4 shows the contact board 10 located in the housing 9 of the main body 2 of the patch device 1. It can be seen in FIG. 4 that the contact board 10 comprises six pins. Two of them, namely a negative voltage pin 21 and a positive voltage pin 22 are equipped with reference signs. For the sake of simplicity, the other four pins are not equipped with reference signs. The negative terminals of both batteries (not shown) of the patch device 1 are connected to the negative voltage pin 21. Likewise, the positive terminals of both batteries (not shown) of the patch device 1 are connected to the positive voltage pin 22.

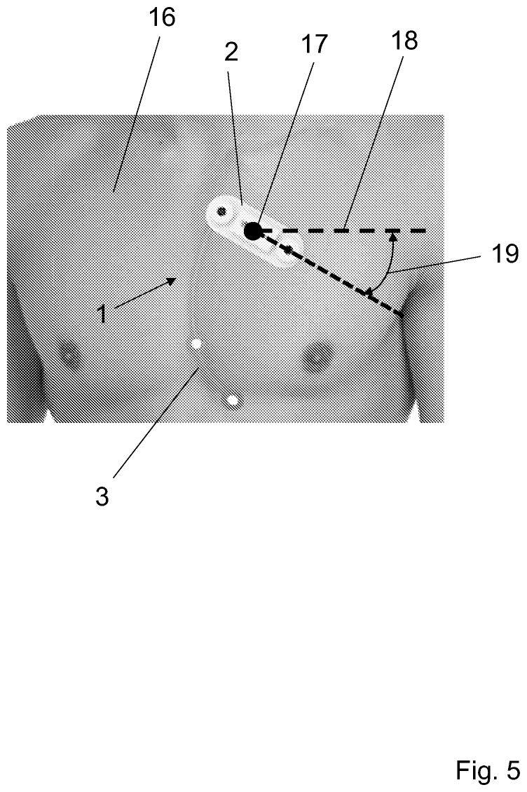

FIG. 5 shows a patch device 1 according to one embodiment placed on a chest 16 of a human being. In particular, one embodiment of a patch device 1 is placed on a chest 16 of a human being in FIG. 5. It can be seen that the patch device 1 comprises a non-transparent main body 2 and a transparent arm portion 3. In FIG. 5 is furthermore indicated a rotation axis 17 around which the patch device 1 is rotated in FIG. 5. In particular, in the use scenario shown in FIG. 5, a rotation angle between the actual orientation of the patch device 1 and a horizontal direction 18 is marked as rotation angle 19. It can be clearly understood from FIG. 5 that the rotation of the patch device 1 around the rotation axis 17 has the effect of better adapting the patch device 1 to the actual form of the chest 16. In other words: In differently shaped chests, the rotation angle of the patch device 1 can be different, namely having a different rotation angle than the rotation angle 19 shown in FIG. 5.

In another use scenario, the patch device 1 is not entirely placed on a chest of a patient but at least partly placed on an arm of the patient. In one particular use scenario, the main body 2 of the patch device 1 is placed on an upper arm of the patient and the arm portion 3 is placed on a chest of the patient. In such a use scenario, the sensor means of the patch device can be used to determine the spatial rotation of the patch device and if a particular spatial rotation is determined, the system deducts from this that the main body of the patch device is placed on the arm of the patient and the arm portion of the patch device is placed on the chest of the patient.

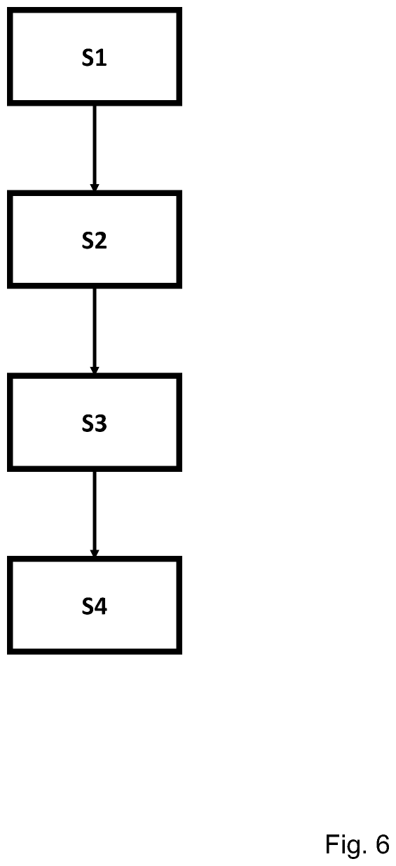

FIG. 6 shows a block diagram visualising a method according to one embodiment. In particular, FIG. 6 shows a series of a baseline calibration step S1, a rotating step S2, a sensor data analysis step S3 and an angle adaptation step S4. In the baseline calibration step S1, a patch device forming part of a system according to an embodiment is provisionally positioned on a body of a living organism, for example on the above-mentioned chest 16, in a baseline position. The baseline position can for example be a position where the main body 2 of the patch device 1 is placed horizontally on the chest, i.e. such that the main body length 11 is oriented in the horizontal direction 18. In this baseline position, a recording of sensor data is carried out wherein this sensor data is referred to as baseline sensor data. This baseline sensor data is recorded, for example in the electronic component of the patch device or yet in a remote device outside the patch.

In the rotating step S2, the patch device 1 is rotated around the rotating axis 17, for example clockwise, until a desired orientation of the patch device 1 is reached. The desired orientation is typically an orientation which corresponds to an ideal positioning of the patch device 1 on the particular chest, for example the chest 16 shown in FIG. 5. The rotating step S2 is followed by the sensor data analysis step S3, during which sensor data supplied by a sensor means of the patch device 1 is analysed in order to determine the spatial orientation of the patch device 1, in particular the rotation angle of the patch device compared to the baseline position. The sensor means is typically arranged in the electronic component of the patch device 1 and typically comprises an accelerometer and/or a gyroscope. During this sensor data analysis step S3, the sensor data recorded at the desired orientation is compared with the baseline sensor data in order to determine the spatial orientation.

FIG. 6 also shows a part of a matching process corresponding to a method for recording an ECG according to one embodiment, namely an angle adaptation step S4. During the angle adaptation step S4, at least one ECG angle is determined based on the spatial orientation determined during the sensor data analysis step S3.

In embodiments, the following steps are carried out in order to make it possible to compensate a rotation of the patch device (these steps are typically carried out before the steps S1 to S4 shown in FIG. 6):

In an ECG data acquisition routine, carried out for a pre-determined number of subjects, the following steps are carried out for each subject:
Patch device is put in standard location, also referred to as baseline position, and one or more of the following leads are measured: Lead I, II, III, aVF, aVL, aVR, V1, V2, V3, V4, V5, V6.
Patch device is placed at different angles of rotation. For each angle of rotation, the leads are measured and stored as the subject's ECG data.

The ECG data of all subjects together form an ECG data set.

Then, an ECG transfer function creation step is carried out which typically comprises the following actions:
From the ECG data set, an ECG transfer function is created which can be applied to map any measured ECG lead measured by the patch device at a particular angle of rotation to the lead values(s) measured at the standard location. This mapping is typically carried out during the above-mentioned matching process.

In embodiments, the following steps are carried out in order to make it possible to determine a QRS axis of an ECG (these steps are typically carried out before the steps S1 to S4 shown in FIG. 6):

In a QRS data acquisition routine, carried out for a pre-determined number of subjects, the following steps are carried out for each subject:
Patch device is put in standard location, also referred to as baseline position, and the QRS axis degree and one of the following three QRS conditions are measured: Normal, Left Axis Deviation (LAD), Right Axis Deviation (RAD).
Patch device is placed at different angles of rotation. For each angle of rotation, the ECG is measured and the measurements are stored as the subject's QRS data.

The QRS data of all subjects together form an QRS data set.

Then, a QRS transfer function creation step is carried out which typically comprises the following actions, per embodiments:
From the QRS data set, a QRS transfer function is created which can be applied to map any measured QRS axis measured by the patch device at a particular angle of rotation to the QRS axis and condition measured at the standard location. This mapping is typically carried out during the above-mentioned matching process.

The above-mentioned routines can be regarded as data acquisition routines for acquiring data sets which can then be used in the above-mentioned systems and methods, in particular in the determination of the spatial orientation of the patch device and/or in the sensor data analysis step and/or in the matching process.

The above-mentioned pre-determined number of subjects can for example be between 10 and 100, preferably between 20 and 80, more preferably between 30 and 60, even more preferably approximately 30. The inventors have found that such numbers of subject offer a good trade-off between routine efficiency on one hand and quality of the acquired data sets.

Furthermore, the following claims are hereby incorporated into the Description of Preferred Embodiments, where each claim may stand on its own as a separate embodiment. While each claim may stand on its own as a separate embodiment, it is to be noted that—although a dependent claim may refer in the claims to a specific combination with one or more other claims—other embodiments may also include a combination of the dependent claim with the subject matter of each other dependent or independent claim. Such combinations are proposed herein unless it is stated that a specific combination is not intended. Furthermore, it is intended to include also features of a claim to any other independent claim even if this claim is not directly made dependent to the independent claim.

It is further to be noted that methods disclosed in the specification or in the claims may be implemented by a device having means for performing each of the respective acts of these methods.

As used herein, the terms “general” and “generally” are intended to account for the inherent degree of variance and imprecision that is often attributed to, and often accompanies, any design and manufacturing process, including engineering tolerances, and without deviation from the relevant functionality and intended outcome, such that mathematical precision and exactitude is not implied and, in some instances, is not possible.

All the features and advantages, including structural details, spatial arrangements and method steps, which follow from the claims, the description and the drawing can be fundamental to the invention both on their own and in different combinations. It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

### LIST OF REFERENCE NUMERALS

1 patch device

2 main body

3 arm portion

4.1, 4.2 electrode contact areas (main body)

5.1, 5.2 electrode contact areas (arm portion)

6.1, 6.2 batteries

7 network of electrode connection lines

8 network of battery connection lines

9 housing

10 contact board

11 main body length

12 main body edge width

13 first horizontal offset

14 second horizontal offset

15 perforation

16 chest

17 rotation axis

18 horizontal direction

19 rotation angle

20 main body electrode contact area distance

21 negative voltage pin

22 positive voltage pin

23 perforation top edge

S1 baseline calibration step

S2 rotating step

S3 sensor data analysis step

S4 angle adaptation step

1. System for monitoring a vital sign of a living organism,
wherein the system comprises a patch device configured to be placed on the living organism,
wherein the patch device comprises a sensor means configured to provide sensor data, wherein
the system is configured to determine a spatial orientation of the patch device via the sensor data when the patch device is placed on the living organism.

2. System for monitoring a vital sign of a living organism,
wherein the system comprises a patch device configured to be placed on the living organism,
wherein
the patch device is configured to be adapted to multiple use scenarios.

3. System according to claim 1, wherein the patch device comprises a disposable patch component and an electronic component, wherein the electronic component comprises the sensor and/or wherein the electronic component is configured to determine said spatial orientation of the patch device.

4. System according to claim 1, wherein the patch device comprises a main body and an arm portion, wherein the main body comprises a housing for the electronic component, and/or wherein the arm portion has an at least partially rounded form, and/or wherein the main body preferably comprises at least one electrode contact area(s), and/or wherein the arm portion comprises at least one electrode contact area(s), and/or wherein the patch device is configured in such a way that the system can acquire an electrocardiogram of the living organism via the electrode area(s).

5. System according to claim 4, wherein the arm portion is configured to be detached from the main body, wherein the patch device comprises a perforation arranged between the arm portion and the main body for detaching the arm portion from the main body.

6. System according to claim 4, wherein the patch device comprises at least two batteries,
wherein the batteries are arranged symmetrically around a center of the patch device, and/or
wherein the batteries are located at two opposite ends of the patch device, and/or
wherein the batteries are connected in parallel, and/or
wherein the patch device comprises a positive voltage pin and a negative voltage pin, wherein the voltage pins are configured to connect to the electronic component, and/or wherein at least two batteries, are connected to the positive voltage pin and/or wherein at least two batteries are connected to the negative voltage pin.

7. System according to claim 4, wherein the patch device comprises a network of electrode connection lines for connecting the electrode contact areas to the electronic component, and/or wherein the electrode connection lines are arranged in a snake coil form on the arm portion.

8. System according to claim 7, wherein the electrode connection lines comprise a mixture of silver and silver chloride, wherein the mixture comprises approximately 50% of silver and approximately 50% of silver chloride.

9. System according to claim 6, wherein the patch device comprises a network of battery connection lines for connecting the batteries to the positive voltage pin and the negative voltage pin, wherein the battery connection lines preferably comprise at least approximately 100% silver, and/or wherein the battery connection lines have a larger cross section surface than the electrode connection lines.

10. System according to claim 4, wherein the patch device comprise(s) comprises a multi-piece protective cover on an adhesive surface on its backside(s).

11. Method for determining a spatial orientation of a patch device for monitoring a vital sign of a living organism during a placing of the patch device on the living organism, wherein the method comprises a sensor data analysis step during which sensor data supplied by a sensor of the patch device is analyzed in order to determine the spatial orientation.

12. Method according to claim 11, wherein
the method comprises a baseline calibration step during which the patch device is provisionally positioned on the living organism in a predefined baseline position and baseline sensor data is recorded, and
the method comprises a rotating step during which the patch device is rotated around an imaginary rotation axis sticking out of the living organism until a desired orientation of the patch device is reached,
wherein the rotating step is followed by the sensor data analysis step,
wherein during the sensor data analysis step, sensor data recorded at the desired orientation is compared with the baseline sensor data in order to determine the spatial orientation.

13. Method for recording an ECG of a living organism, wherein the method comprises a method for determining a spatial orientation of a patch device according to claim 11,
wherein
the method for recording comprises a matching process during which the spatial orientation determined during the sensor data analysis step is used for matching obtained electrocardiogram signals with pre-recorded electrocardiogram data, and/or
the method for recording comprises an angle adaptation step during which at least one ECG angle is/are determined based on the spatial orientation determined during the sensor data analysis step, and/or
the method for recording comprises a lead renaming step during which at least one ECG lead is/are renamed based on the spatial orientation determined during the sensor data analysis step, and/or
the method for recording comprises a chest-arm-detection step during which, based on the spatial orientation determined during the sensor data analysis step, it is automatically detected whether the patch device is placed on a chest or on an arm of a human being.

14. Computer program comprising instructions which, when the program is executed by a computer, cause the computer to carry out a method according to claim 11.

15. Computer-readable medium comprising a computer program according to claim 14.

16. Method for using a system according to claim 4, wherein the method comprises attaching the main body of the patch device to an upper arm of a patient and attaching the arm portion of the patch device to a chest of the patient, wherein the main body comprises the electronic component.

17. System according to claim 7, wherein the electrode connection lines comprise a mixture of silver and silver chloride, wherein the mixture comprises approximately 80% of silver and approximately 20% of silver chloride.

18. System according to claim 7, wherein the electrode connection lines comprise a mixture of silver and silver chloride, wherein the mixture comprises approximately 95% of silver and approximately 5% of silver chloride.
